(19) United States
(12) Patent Application Publication
(10) Pub. No.: US 2017/0290630 A1
(43) Pub. Date: Oct. 12, 2017

(54) SURGICAL ROBOT SYSTEM FOR USE IN AN MRI

(71) Applicant: ENGINEERING SERVICES INC., Toronto (CA)

(72) Inventors: Andrew A. GOLDENBERG, Toronto (CA); Yi YANG, Toronto (CA); Liang MA, Markham (CA)

(21) Appl. No.: 15/092,230

(22) Filed: Apr. 6, 2016

(51) Int. Cl. A61B 34/20; G06K 7/10; A61B 90/00; A61B 5/055; A61B 5/00

(52) U.S. Cl. CPC A61B 34/20; A61B 5/055; A61B 5/0046; A61B 90/08; G06K 7/10366

(57) ABSTRACT

A surgical robot assembly for use with an MRI includes a surgical robot, a controller, cables, a dedicated room ground and a filter. The surgical robot includes at least one ultrasonic motor and all the motors therein are ultrasonic motors. The controller is spaced from the surgical robot and is positioned outside the MRI room. The controller has at least one analog output; at least one digital input, at least two digital output, and at least one encoder reader channel. The cables are operably attaching the motors of the surgical robot to the controller and are RF shielded. The cables are operably connected to the dedicated room ground. The filter is operably connected to the cables which are operably connected between the motors of the surgical robot and the controller and the filter has a cut off frequency tuned to the MRI.

### FIELD OF THE DISCLOSURE

This disclosure relates to medical robot systems and in particular a medical robot system for use in an MRI.

### BACKGROUND

It is well known that medical resonance imaging (MRI) devices have excellent soft tissue resolution and generate minimal radiation hazard. Because of these advantages MRI-guided robotic-based minimally invasive surgery has become an important surgical tool.

There is a number of surgical robots currently in use but not all are compatible with an MRI. For example the Intuitive Surgical robot called the da Vinci™ is not compatible with an MRI. In contrast the Innomotion™ robot arm (Innomedic), the NeuroArm™ robot (University of Calgary), and the MRI-P™ robot (Engineering Services Inc.) are all MRI-compatible. However, even those robots which are MR compatible may not be able to be operated during MRI operation of scanning.

The main reasons that the robots have not been widely used in the MRI environment are MRI incompatibility and more particularly limitations of the real-time intra-operative imaging.

### SUMMARY

A surgical robot assembly for use with an MRI includes a surgical robot, a controller, cables, a dedicated room ground and a filter. The surgical robot includes at least one ultrasonic motor and all the motors therein are ultrasonic motors. The controller is spaced from the surgical robot and is positioned outside the MRI room. The controller has at least one analog output; at least one digital input, at least two digital output, at least one encoder reader channel. The cables are operably attaching the motors of the surgical robot to the controller and are RF shielded. The cables are operably connected to the dedicated room ground. The filter is operably connected to the cables which are operably connected between the motors of the surgical robot and the controller and the filter has a cut off frequency tuned to the MRI.

The surgical robot may include a plurality of motors and the controller may include a plurality of analog outputs and the plurality of motors may be operably attached to the same controller.

The controller may be a USB4 controller.

The cables may be shielded with copper tube sleeves.

The surgical robot may include a plurality of motors and each motor has a cable between the motor and the controller and a plurality of cables may be bundled together in a copper tube sleeve. The plurality of motors may be operably attached to the same controller.

The dedicated ground may be attached to the cables and attached to a wall of the MRI room.

The filter may be a low pass filter.

The MR scanner may be a Philips 3.0T MR scanner and the low pass filter may have 3 DB cut off frequency at 3.2 MHz.

The filter may be a SPECTRUM CONTROL-56-705-003-FILTERED D Sub-connector.

Further features will be described or will become apparent in the course of the following detailed description.

### BRIEF DESCRIPTION OF THE DRAWINGS

The embodiments will now be described by way of example only, with reference to the accompanying drawings, in which:

FIG. 1 is a perspective view of a prior art surgical robot for use in an MRI;

FIG. 2 is a schematic diagram of the connection between the ultrasonic motors and the computer in the prior art surgical robot of FIG. 1;

FIG. 3 is a perspective view of an improved surgical robot for use in an MRI;

FIG. 4 is perspective view of the improved surgical robot similar to that shown in FIG. 3 but showing the MRI, an MRI table and an MRI room wall;

FIG. 5 is a schematic diagram of the connection between the ultrasonic motors and the computer of the improved surgical robot;

FIG. 6 is a schematic diagram of the connection between a plurality of ultrasonic motors and the computer of the improved surgical robot;

FIGS. 7A and 7B are cross sectional views of the shielding sleeve and cables of the improved surgical robot of FIG. 3, wherein FIG. 7A shows a single motor cable and a single encoder cable in a shielding sleeve and FIG. 7B shows a plurality of motor cables and a plurality of encoder cables in a single shielding sleeve;

FIGS. 8A, 8B, and 8C is a sequence of MRI images of apiece of meat of a 2-D FGRE (axial) taken using the prior art surgical robot, wherein FIG. 8A is without the motor, FIG. 8B is with the motor powered on without motion and FIG. 8C is with the motor moving;

FIGS. 9A, 9B, and 9C is a sequence of MRI images of a piece of meat of a 2-D FSE T2 (axial) taken using the surgical robot with shielded cables, wherein. FIG. 9A is without the motor, FIG. 9B is with the motor powered on without motion and FIG. 9C is with the motor moving;

FIGS. 10A, 10B, and 10C is a sequence of MRI images of a piece of meat of a 2-D FGRE (axial) taken using the surgical robot with shielded cables, wherein FIG. 10A is without the motor, FIG. 10B is with the motor powered on without motion and FIG. 10C is with the motor moving;

FIGS. 11A and 11B is a sequence of MRI images of a phantom of a 2-D FSE T2 (axial) taken using the surgical robot with a USB4 controller and shielded cables, wherein FIG. 11A is without the motor and FIG. 11B is with the motor moving;

FIGS. 12A and 12B is a sequence of MRI images of a piece of meat of a 2-D FGRE (axial) taken using the improved surgical robot assembly of FIG. 3, wherein FIG. 12A is with the motor powered on without motion and FIG. 12B is with the motor moving; and

FIGS. 13A, 13B and 13C is a sequence of MRI images of a small watermelon of a 2-D FGRE (axial) taken using the improved surgical robot assembly of FIG. 3, wherein FIG. 13A is with the motor powered on without motion, FIG. 13B is with the turret module of the surgical robot moving and FIG. 13C is with surgical tool moving.

### DETAILED DESCRIPTION

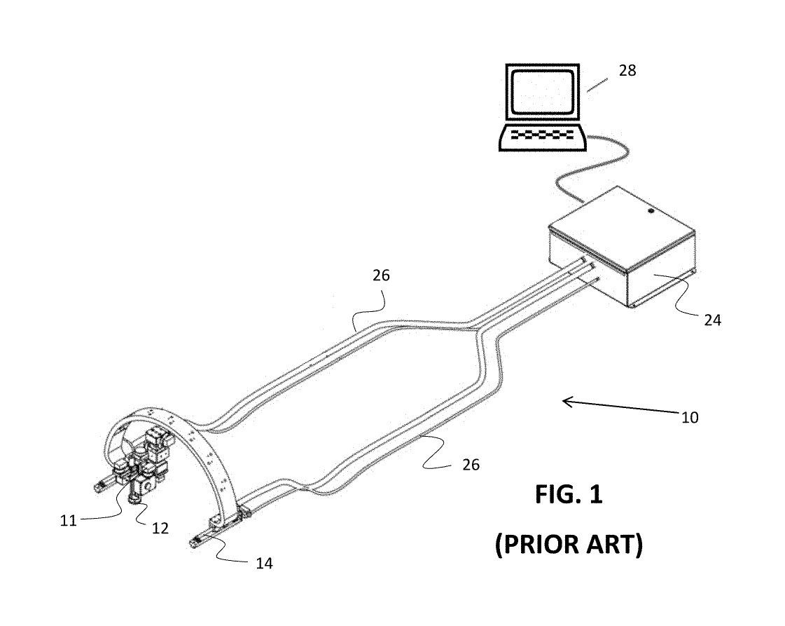

Referring to FIG. 1, a prior art surgical robot system for use in an MRI is shown generally at 10. By way of example, surgical robot system 10 includes a six-degree of freedom surgical robot 11 that uses ultrasonic motors. The surgical robot 11 has a surgical tool 12 attached thereto and is moveable on a pair of rails 14. The surgical tool 12 may include an ultrasonic motor. The rails 14 typically will include a pair of ultrasonic motors for moving the surgical robot 10 along the rails.

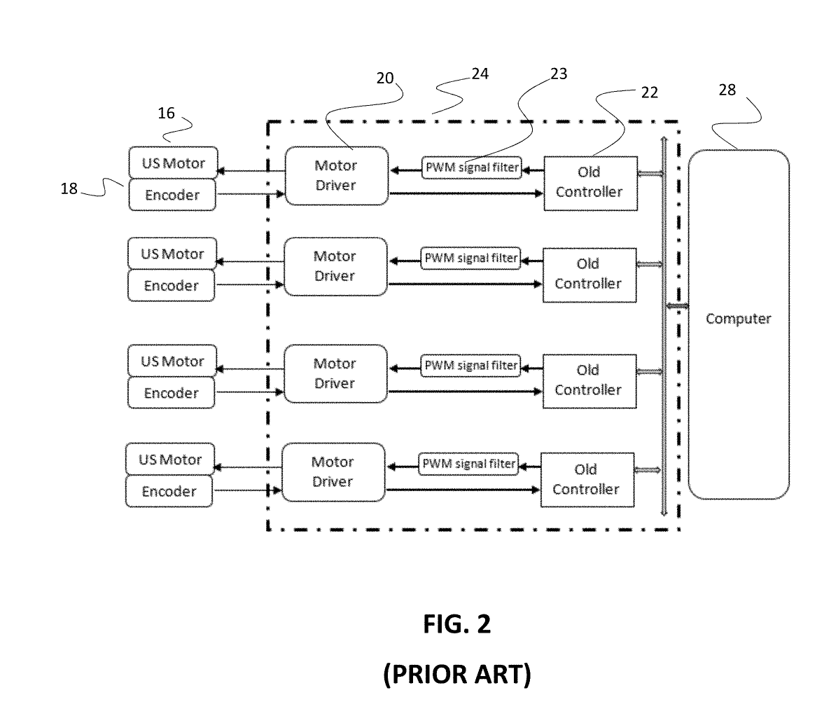

Referring to FIG. 2, the prior art surgical robot system 10 shown in FIG. 1 includes a plurality of ultrasonic motors 16. Each ultrasonic motor 16 is operably connected to an encoder 18. Each ultrasonic motor 16 and encoder 18 is operably connected to a motor driver 20. The motor driver 20 is operably connected to a controller 22 which includes a PWM (pulse width modulation) and a PWM signal filter 23. The controllers 22 and the motor drivers 20 are located in an electronics box 24 and are connected to the motors 16 and encoders 18 of the surgical robot 10 with cables 26. The electronic cables 26 are shielded with an aluminium membrane. The controllers 22 in the electronic box 24 are operably connected to a computer 26. The prior art robot assembly shown in FIGS. 1 and 2 is described in detail in U.S. patent application Ser. No. 14/619,978, filed Feb. 11, 2015 entitled “Surgical Robot” with Goldenberg et al. as inventors.

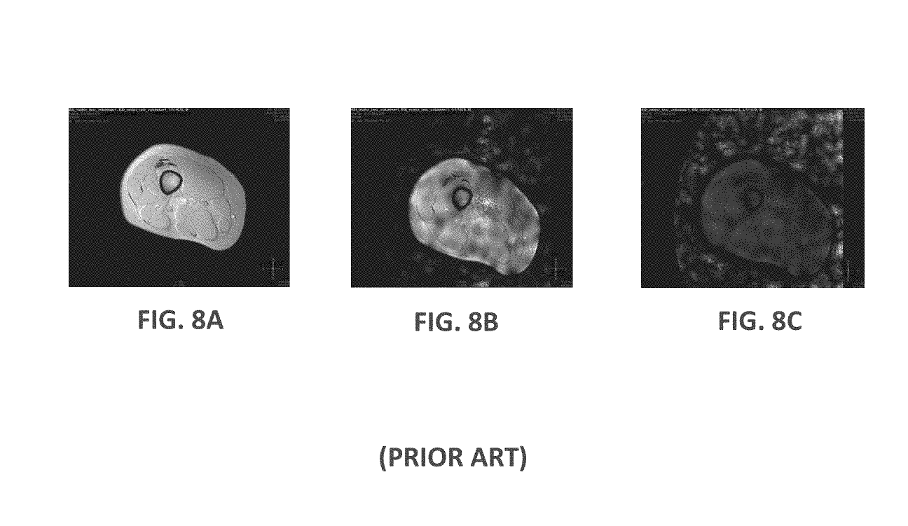

Prior art surgical robot system 10 is compatible with an MRI but if the motors are powered on the MR image is degraded in the form of noise and artifacts, the degradation of the MR image is increased if the motors are moving. This can clearly be seen in the MR images shown in FIG. 8 wherein FIG. 8A is an MR image without motor, FIG. 8B is with the motor powered on without motion and FIG. 8C is with the motor moving.

The Ultrasonic Motors (USM) motion is generated mechanically by contact friction not electro-mechanically; there are no ferromagnetic parts. Thus, ultrasonic motors are considered suitable for the MRI environment, and may be used in devices working in or in the vicinity of MRI bore. However, the motor driver electronics that controls the motor motion generally produce noise on the MR images. Typically when the motor driver electronics are powered on they generates RF noise. In addition the motor/encoder cables may act as antennas emitting RF signals that interfere with the MR imaging process. This interference is in the form of noise and artifacts on the MR images. The noise and artifact constrain the use of ultrasonic motors in the MRI environment. In the prior art robot 10 shown in FIG. 1 the ultrasonic motors operation (motion) and MR imaging (scanning) are intercalate. Although widely accepted this solution limits operational functionality. Alternatively the ultrasonic motor drivers are “tuned-up” to the driver in the MRI firing sequence. The tune-up activates the driver when the scanning sequence is at rest, and vice-versa. This method is cumbersome to implement.

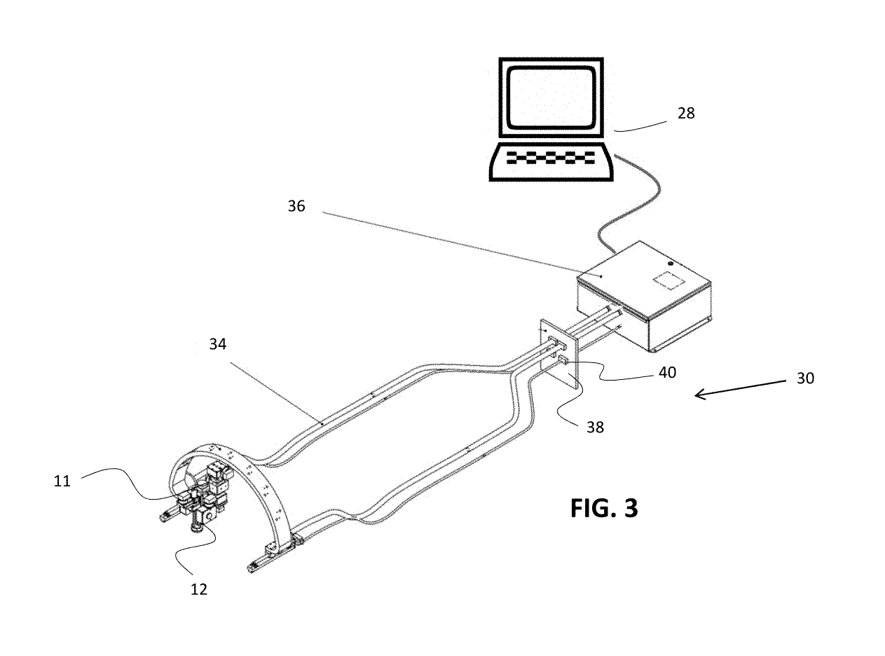

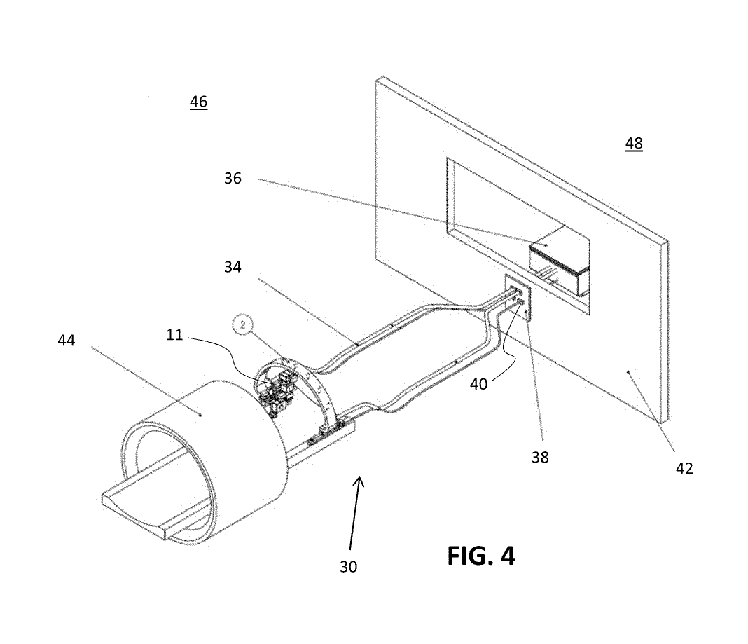

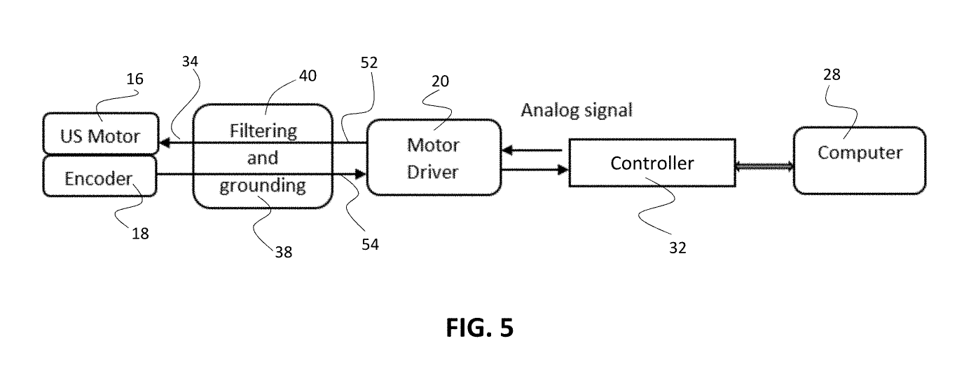

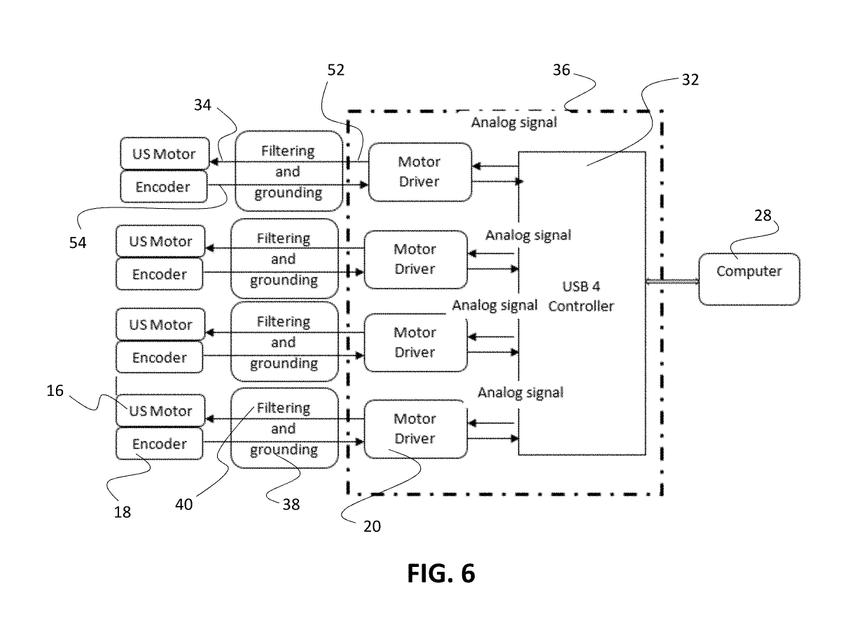

The improved surgical robot system for use with an MRI is described below with reference to FIGS. 3 to 6. The improved surgical robot system 30 greatly decreases the noise and artifacts on the MRI image when the ultrasonic motors are in use. Referring to FIG. 3, an improved surgical robot system is shown generally at 30. The improved surgical robot system 30 is similar to that shown in FIG. 1. However the connection of each of the surgical robot 11, surgical tool 12 and pair of rails 14 to the computer 28 is different. The ultrasonic motors in each of the surgical robot 11, surgical tool 12 and rails 14 are operably connected to a controller 32 (shown in FIGS. 5 and 6) with cables 34. The controllers 32 are in an electronic box 36. The controllers 32 in the electronic box 36 are operably connected to the computer 28. The electronic box is made of aluminum. The cables 34 are operably connected to a dedicated room ground 38 and filter 40. The room ground 38 is connected to the MRI room wall 42. The MRI machine 44 and robot 11 are situated inside the MRI room 46 and the electronic box 36 and the computer 28 are situated outside of the MRI room in a control room 48. As is well known to those skilled in the art the MRI room is shielded to avoid RF noise. It will be appreciated by those skilled in the art that robot 11 is shown herein by way of example only and that other surgical robot that uses ultrasonic motors could also be used.

The connection for each ultrasonic motor 16 to the computer 28 is shown in FIG. 5 and the connection of a plurality of ultrasonic motors 16 to the computer 28 is shown in FIG. 6. Controller 32 includes at least one encoder reader channel, at least one digital input port, at least two digital output port and at least one analog output port. It will be appreciated by those skilled in the art that since the controller includes at least one analog output the controller has a digital to analog converter included therein.

Preferably the controller includes a plurality of analog output ports, a plurality of encoder readers, and a plurality of digital output ports. By way of example the USB4™ produced by US Digital is used in controller 32. The USB4™ includes four (4) channels of encoder readers, eight (8) digital outputs, four (4) analog outputs, eight (8) digital inputs, four (4) analog inputs. Each ultrasonic motor 16 of the surgical robot 30 uses one channel encoder reader, one analog output, one digital input and two digital output. Therefore four ultrasonic motors are controlled by one USB4™. Since the surgical robot 11 that is shown by way of example includes nine ultrasonic motors in the improved surgical robot system 30 described herein two USB4 controllers are used as well as a dedicated controller used in associated with one of the specific motor. Surgical robot 11 includes eight Shinsei Ultrasonic Motor and one Korean motor PUMR40E Model: PUMR40E-DN™ this motor has a dedicated controller which is housed in the electronic box 36. The dedicated controller has similar features to those described above but for use with a single motor.

The USB4 is connected through a USB port with a PC. In practice the controller 32 or more specifically the USB4s and the dedicated Korean motor controller together with the computer 28 operate together to control the ultrasonic motors 16. The USB4 and the dedicated Korean controller each provide an analog signal that controls the USM speed. In such configuration the USB4 and the PC operate together as the motor controller. It will be appreciated by those skilled in the art that the number of controllers 32 or controllers with a plurality of analog inputs will be determined by the number of motors in the surgical robot. Accordingly this may be scaled up or down depending on the number of motors.

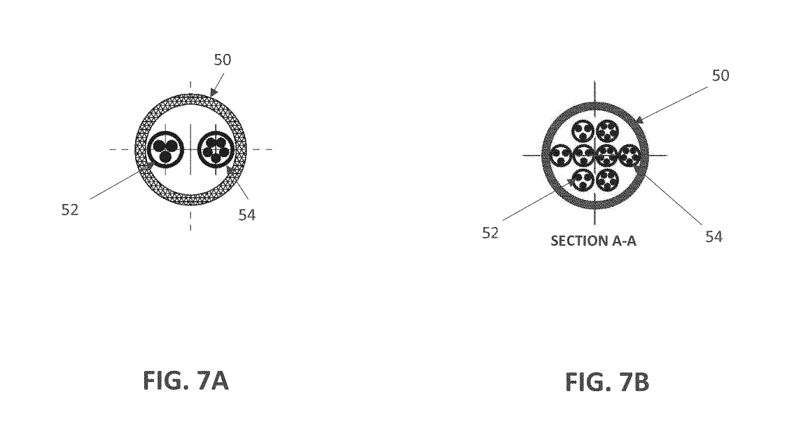

The cables 34 connecting the motors 16 and encoders 18 to the motor drivers 20 are provided with RF shielding. By way of example, a tin copper tube sleeve 50 is used. As shown in FIG. 7A there is a separate motor cable 52 that operably connects the US motor 16 to the controller and a separate encoder cable 54 that operably connects the encoder 18 to the controller 32. A plurality of cables 34 may be bundled together in one tin copper tube sleeve 50 as shown in FIG. 7B. It will be appreciated by those skilled in the art that alternate RF shielding materials could also be used. Tin copper shielding was chosen as it currently provides a good balance between shielding results and cost. The requirement of RF shielding material is that it must have good conductivity of electricity. Other alternatives would be copper, galvanized steel, silver or gold. However some of these options are unlikely due to the cost of materials. The tin-copper sleeve used herein by way of example is made up of a plurality of small tin copper wires that are coven together.

The electronic box 36 and the shielding tubes 50 are connected to the room ground 38. It has been observed that the grounding significantly improves the effectiveness of the shielding provided by the tin copper tube sleeve 50. Further it has been observed that the grounding of the shielding tubes and electronic box to the ground of a wall power outlet does not significantly reduce the RF noise. A dedicated ground 38 of the MRI room is used for grounding the shielding and electronic box.

It has been observed that typically MRI machines are sensitive to signals of a specific frequency range. For example, Philips 3.0T MR scanner is sensitive to 80 MHz and higher signals. A low pass filter 40 is added to reduce the noise at this and higher frequencies. A “low pass” filter is used such that only low frequency signals can pass. As is well known in the art MRI machines are very sensitive to their resonant frequency. Usually the resonant frequency for an MRI machine is between 60 and 80 Mhz.

Ideally the low pass filter 40 should eliminate any noise signal affecting the MRI machine resonant frequency. The cut off frequency of the low pass filter depends on the specific a MRI machine and noise level. Preferably the low pass filter 40 provides at least −20 DB reduction at the MRI resonant frequency. Preferably the cut off frequency of the low pass filter 40 is much lower than MRI resonant frequency. By way of example a SPECTRUM CONTROL-56-705-003-FILTERED D Sub-connector is used for filtering. This sub-connector has a built-in lowpass filter with the 3 DB cut off frequency at 3.2 MHz. The low pass filter 40 is operably connected the MRI dedicated room ground 38.

Images obtained from an MR scanner show the surprising and significant improvement obtained with the improved surgical robot assembly 30. More specifically FIGS. 8A, 8B and 8C shows a sequence of MRI images of a piece of meat of a 2-D FGRE (fast gradient recalled echo sequences) (axial) taken using the prior art surgical robot, wherein FIG. 8A is without the motor, FIG. 8B is with the motor powered on without motion and FIG. 8C is with the motor moving. These images clearly show that the prior art robot cannot be used concurrently with MR scanning.

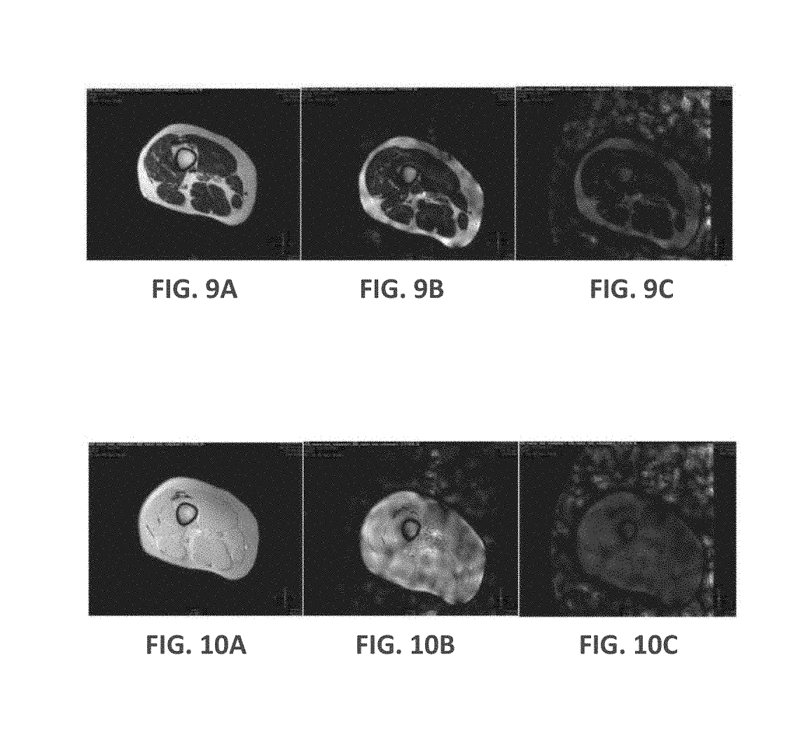

FIGS. 9A, 9B, and 9C is a sequence of MRI images of a piece of meat of a 2-D FSE T2 (fast spin echo with T2 weighting sequences) (axial) taken using the surgical robot with shielded cables, wherein FIG. 9A is without the motor, FIG. 9B is with the motor powered on without motion and FIG. 9C is with the motor moving. FIGS. 10A, 10B, and 10C show a sequence of MRI images of a piece of meat of a 2-D FGRE (axial) taken using the surgical robot with shielded cables, wherein FIG. 10A is without the motor, FIG. 10B is with the motor powered on without motion and FIG. 10C is with the motor moving. These images clearly show that when the prior art robot assembly with new cable shielding of tin copper tube sleeve is used, by observation, the images in the FSE T2 sequences show images having small artifact and medium noise degradation and in the FGRE sequence images having medium artifact and large noise degradation.

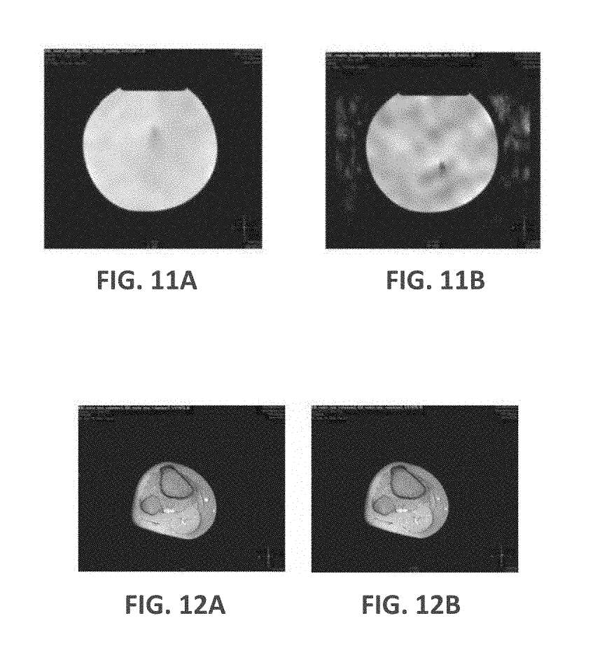

FIGS. 11A and 11B shows a sequence of MRI images of a phantom of a 2-D FSE T2 (axial) taken using the surgical robot with a USB4 controller and shielded cables, wherein FIG. 11A is without the motor and FIG. 11B is with the motor moving. These images show medium artifact and large noise degradation.

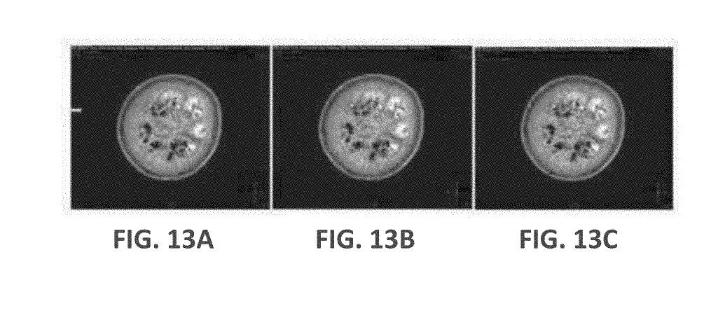

In contrast the images shown in FIGS. 12 and 13 taken with the improved surgical robot 20 show little degradation. More specifically FIGS. 12A and 12B show a sequence of MRI images of a piece of meat of a 2-D FGRE (axial) taken using the improved surgical robot assembly of FIG. 3, wherein FIG. 12A is with the motor powered on without motion and FIG. 12B is with the motor moving. FIGS. 13A, 13B and 13C show a sequence of MRI images of a small watermelon of a 2-D FGRE (axial) taken using the improved surgical robot assembly of FIG. 3, wherein FIG. 13A is with the motor powered on without motion, FIG. 13B is with the turret module of the surgical robot moving and FIG. 13C is with surgical tool moving. By observation FIGS. 12 and 13 show that the quality of MR images appears not to be affected; that is, with reference to the images no significantly interfering frequencies were observed, other forms of noise were not observed, significant deterioration of the images was not observed, and image shifts were also not observed.

Generally speaking, the systems described herein are directed to surgical robots. Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein the “operably connected” or “operably attached” means that the two elements are connected or attached either directly or indirectly. Accordingly the items need not be directly connected or attached but may have other items connected or attached therebetween.

What is claimed is:

1. A surgical robot assembly for use with an MRI scanner that is housed in an MRI room comprising:
a surgical robot with at least one ultrasonic motor wherein all the motors therein are ultrasonic motors;
a controller spaced from the surgical robot and positioned outside the MRI room, the controller having at least one analog output, at least one digital input, at least two digital output, and at least one encoder reader channel;
cables operably attaching the motors of the surgical robot to the controller, the cables being RF shielded;
a dedicated room ground and the cables which are operably connected between the motors of the surgical robot and the controller are operably connected thereto; and
a filter operably connected to the cables which are operably connected between the motors of the surgical robot and the controller, the filter having a cut off frequency tuned to the MRI.

2. The surgical robot assembly of claim 1 wherein the surgical robot includes a plurality of motors and a plurality of encoders and the controller includes a plurality of analog outputs and a plurality of encoder reader channels and the plurality of motors and the plurality of encoders are operably attached to the same controller.

3. The surgical robot assembly of claim 2 wherein the controller is a USB4 controller.

4. The surgical robot assembly of claim 1 wherein the cables are shielded with copper tube sleeves.

5. The surgical robot assembly of claim 4 wherein the surgical robot includes a plurality of motors and a plurality of encoders and each motor has a cable between the motor and the controller and each encoder has a cable between the encoder and the controller and a plurality of cables are bundled together in a copper tube sleeve.

6. The surgical robot assembly of claim 5 wherein the plurality of motors are operably attached to the same controller.

7. The surgical robot assembly of claim 6 wherein the controller is a USB4 controller.

8. The surgical robot assembly of claim 1 wherein the dedicated ground is attached to the cables and attached to a wall of the MRI room.

9. The surgical robot assembly of claim 1 wherein the filter is a low pass filter.

10. The surgical robot assembly of claim 9 wherein the MR scanner is a Philips 3.0T MR scanner and the low pass filter has a 3 DB cut off frequency at 3.2 MHz.

11. The surgical robot assembly of claim 9 wherein the filter is a SPECTRUM CONTROL-56-705-003-FILTERED D Sub-connector.
